(19) United States
(12) Patent Application Publication
(10) Pub. No.: US 2003/0072782 A1
(43) Pub. Date: Apr. 17, 2003

(54) Combined insec repellent and suncreen composition

(76) Inventors: Michael Christopher Friel, New South Wales (AU); Rachel Louise Ahyong, New South Wales (AU); Ian Andrew Thompson, New South Wales (AU)

Correspondence Address: FISH & RICHARDSON P.C., 45 ROCKEFELLER PLAZA, SUITE 2800, NEW YORK, NY, 10111, US

(21) Appl. No.: 09/889,031

(22) PCT Filed: Jan. 11, 2001
(86) PCT No.: PCT/GB00/00068

(51) Int. Cl. A01N 25/00; A61K 7/42

(52) U.S. Cl. 424/405; 424/59

(57) ABSTRACT

A combined insect repellent and sunscreen composition is disclosed including 3-9 % by weight in total of at least two emulsifiers, based on the total weight of the composition. The composition preferably comprises titanium dioxide as the inorganic compound and N, N-diethyl-m-toluamide and dipropyl pyridine-2,5-dicarboxylate as insect repellents. A method of manufacturing a sunscreen composition is also disclosed. The composition including one or more insect repellents and one or more sunscreening agents, is the form of an emulsion having an oil phase and a water phase and is manufactured by preparing the oil phase and the water phase and combining to form an emulsion prior to the addition of at least one inorganic compound used as a sunscreening agent.

### TECHNICAL FIELD

This invention relates to insect repellent and sunscreen compositions and in particular to combined insect repellent and sunscreen compositions that have both effective repellency and sunscreening properties.

### BACKGROUND ART

The prior art is replete with insect repellent compositions and sunscreen compositions. Whilst these compositions are separately effective, it is desirable to provide an effective combined insect repellent and sunscreen composition.

Surprisingly, it has now been found that to produce a stable, effective combined insect repellent and sunscreen composition requires a carefully selected use of emulsifiers.

### DISCLOSURE OF INVENTION

Accordingly, the present invention consists in a sunscreen composition including one or more insect repellents and one or more UV sunscreening agents characterised in that the composition includes 3-9% by weight in total of at least two emulsifiers, based on the total weight of the composition. The inventors have found that with appropriate emulsifiers, the sunscreen agent in combination with insect repellent give a composition that is stable and effective with respect to SPF.

One or more inorganic compounds are incorporated in the composition of the invention as a sunscreening agent. The preferred inorganic compounds are titanium oxide and zinc oxide. For these compounds, the particle size can be selected to scatter light in the UW range whilst transmitting light in the visible range thereby remaining transparent on the skin. This is highly desirable from a cosmetic point of view. Micronised particles, that is those particles less than 100 nm in size, give optimal performance. Micronised titanium dioxide is most preferred for the composition of the invention. The concentration of inorganic compound may be in the range of 1-5% by weight based on the total weight of the composition, preferably 2-4% by weight and more preferably 3% by weight.

One or more insect repellents are included in the composition. The repellents are chosen for repellency of flying or biting insects and for low skin irritancy. Suitable repellents include N,N-diethyl-m-toluamide (DEET), dipropyl pyridine-2,5-dicarboxylate, pyrethrins, dimethyl phthalate, 2,3:4,5-bis(2-butylene)tetrahydrofurfuryl, citronella, geraniol, lemon grass oil, eugenol, p-menthane-3,8-diol, ethylbutyl acetylamino propionate, 1-piperidinecarboxylic acid and 2-(2-hydroxyethyl)-ester1-methylpropyl-ester.

These can be combined with synergists such as piperonyl butoxide and N-(2-ethylhexyl)-8,9,10-trinorborn-5-ene-2,3-dicarboximide.

DEET and dipropyl pyridine-2,5-dicarboxylate are the preferred repellents.

The total amount of insect repellent in the composition may be 4-20% by weight based on the total weight of the composition, preferably 4-15% by weight and more preferably 5-10% by weight.

The composition may include one or more other UV sunscreening agents. These are generally organic compounds which absorb a specific range of UV radiation. Suitable sunscreening agents include octyl methoxycinnamate, oxybenzone, amino benzoic acid, Cinoxate, DEA-methoxycinnamate, Digalloyl, Dioxybenzene, Padimate O, Ethyl dihydroxypropyl p-aminobenzoate, octyl salicylate, glyceryl aminobenzoate, Homosalate, Urocanic acid, isopropylbenzyl salicylate, menthyl anthranilate, octocrylene, Sulisbenzone and its sodium salt and triethanolamine salicylate.

A combination of octylmethoxycinnamate and oxybenzone is most preferred. Each sunscreening agent is preferably incorporated in the composition in an amount of 3-10% by weight based on the total weight of the composition.

The composition is prepared in the form an emulsion. Accordingly, a second aspect of the invention consists in a sunscreen composition further including, by weight, based on the total weight of the composition,

7% in total emulsifier,

up to 5%, preferably 1-5%, more preferably 3% film former,

up to 0.25%, preferably 0.05-0.25%, more preferably 0.15% thickener,

up to 0.3%, preferably 0.1-0.3%, more preferably 0.15% neutraliser,

up to 0.3%, preferably 0.1-0.3%, more preferably 0.2% chelating agent and up to 2.5% of at least one of preservative, perfume and moisturiser.

The choice of emulsifier will depend on the insect repellents and sunscreening agents selected. More than two emulsifiers may be included. An emulsion is most accurately defined as a dispersion of liquid droplets in a second immiscible liquid. Dispersions may be formed temporarily through agitation of the two immiscible liquids, however, resolution of the emulsion is usually rapid and complete unless a stabilising additive or emulsifier is used.

Emulsions usually consist of water or an aqueous solution as one immiscible phase and some organic liquid, or “oil”, as the other phase. When the oil is dispersed in the aqueous phase the emulsion is called oil in water (o/w) or alternatively, if the aqueous phase is dispersed in the oily phase the emulsion is described as water in oil (w/o). An emulsifying agent is usually required to stabilise the emulsion. Such agents are ordinarily large molecules of which the greatest part of the molecule is non-polar (for solubility in the oil phase) and a smaller part is polar (for orientation and solubility into the water phase).

Typical properties of oil in water emulsions include: creamy feel, mixing readily with water and high SPF efficacy. The composition of the invention is preferably in the form of an oil in water emulsion.

An example of a suitable emulsifying system includes fatty acid ethoxylates such as glycerol monostearate, fatty alcohol ethoxylates such as ethoxy (20) stearyl alcohol, fatty alcohols such as C₁₆₋₁₈ fatty alcohols, and blends of fatty alcohol ethoxylates with alkyl phenol ethoxylates such,as a blend of cetostearyl alcohol and PEG stearate. A wide range of other emulsifiers also appear useful for this purpose.

The composition optionally includes a film former. The preferred film former is Tricontanyl PVP.

The composition may include thickeners, chelating agents and pH adjusting agents as required. These are readily known to the person skilled in the art. Suitable thickeners include acidic acrylates such as carboxyl polymethylene, and cellulose based thickeners such as methyl cellulose, guar gum, sodium alginate and sodium carboxymethyl cellulose. A suitable chelating agent is disodium EDTA. Triethanolamine may be used as a neutraliser as if required.

The person skilled in the art will recognise that perfumes, emollients and moisturisers may be included to satisfy organoleptic requirements.

Preservatives may also be used as required. These are readily known to the person skilled in the art.

The inventors have found that in preparing an emulsion, the order of addition of ingredients affects the SPF of the final composition.

Accordingly, a third aspect of the invention consists in a method of manufacturing a sunscreen composition including one or more insect repellents and one or more UV sunscreening agents, the composition being in the form of an emulsion having a water phase and an oil phase characterised in that the water phase and oil phase are prepared and combined to form an emulsion prior to the addition of at least one inorganic compound which is used as a sunscreening agent.

In a fourth aspect, the invention consists in a method of manufacturing a sunscreen composition including the steps of:

(a) preparing a water phase including water and thickener

(b) preparing an oil phase including at least two emulsifiers, at least one insect repellent, at least one organic sunscreen and optionally, a film former,

(c) combining said water phase and oil phase to form an emulsion; and

(d) adding at least one inorganic compound as a sunscreening agent.

In a fifth aspect, the invention consists in a sunscreening composition manufactured according to the methods described above.

The invention will now be further described with reference to a number of examples.

Modes for carrying Out the Invention

| | Formula 1 | Formula 2 | Formula 3 |
|---|---|---|---|
| Ingredients | w/w % | w/w % | w/w |
| DEET | 7 | 7 | 7 |
| MGK-326 | 2.8 | 2.8 | 2.8 |
| Parsol MCX | 7.5 | 9.0 | 9.0 |
| Oxybenzone USP | 3 | 5 | 5 |
| Tioveil AQ-G | 7.5 | 10 | 7.5 |
| Cithrol GMS A/S | 1.5 | 1.5 | 1.5 |
| Volpo S20 | 2 | 2 | 2 |
| Crodacol CS70 | 1.75 | 1.75 | 1.75 |
| Polawax GP 200 | 1.75 | 1.75 | 1.75 |
| Antaron WP-660 | 3 | 3 | 3 |
| (Tricontanyl PVP) | | | |
| Silicone DC 200/500 | 0.3 | 0.3 | 0.3 |
| Carbopol 940 | 0.15 | 0.15 | 0.15 |
| Aloe Vera powder 1:200 | 0.01 | 0.01 | 0.01 |
| Disolvine Na2 | 0.2 | 0.2 | 0.2 |
| (disodium EDTA) | | | |
| Triethanolamine 85% | 0.15 | 0.15 | 0.15 |
| Germaben II-E | 1 | 1 | 1 |
| Perfume Kokoda 6463 | 0.3 | 0.3 | 0.3 |
| Water | 60.09 | 54.09 | 56.59 |
| | 100 | 100 | 100 |

The ingredients listed above are further described below in Table 1.

TABLE 1

| INGREDIENT (SUPPLIER) | PURPOSE |
|---|---|
| DEET (MGK) | Mosquito repellent |
| N,N-diethyl-m-toluamide | |
| MGK-326 (MGK) | Fly repellent |
| dipropyl pyridine-2,5-dicarboxylate 99% | |
| Parsol MCX (Givaudan) | UVB filter, |
| octyl methoxycinnamate 98% | organic sunscreen |
| Benzophenone-3 (Aceto Corp.) | UVA/B filter, |
| oxybenzone 98% | organic sunscreen |
| Tioveil AQ | UVA/B filter, |
| micronised titanium dioxide 40% | organic sunscreen |
| Cithrol GMS A/S (Croda) | emulsifier |
| glycerol monostearate | |
| Volpo S20 (Croda) | emulsifier |
| ethoxy (20) stearyl alcohol | |
| Crodacol CS70 (Croda) | emulsifier |
| cetoaryl alcohol 35/65 | |
| Polawax GP 200 (Croda) | emulsifier |
| blend of cetostearyl alcohol and | |
| PEG stearate | |
| Antaron WP-660 (ISP) | film former |
| 2-pyrrolidinone, 1-ethenyl | |
| polymer with 1-triacontene | |
| Silicone DC 200/500 (Dow Corning) | emollient |
| silicone oil 200/500 | |
| Carbopol 940 (B F Goodrich) | thickener |
| carboxyl polymethylene | |
| Aloe Vera powder 1:200 | moisturiser |
| Sequestrene NA2 | chelating agent |
| disodium EDTA | |
| Triethamolamine H/H (Union Carbide) | neutraliser |
| Germaben II-E | preservative |
| Kokoda 6463 | perfume |
| Water | diluent |

Preparation

A water phase is prepared by adding water to a clean, dry mixing vessel and stirring to create a vortex. Carbopol powder is sprinkled into the vortex and the mixture heated to 75-80° C.

In a separate vessel, an oil phase is prepared by adding emulsifiers, film former, mosquito and fly repellent i.e. Cithrol GMS A/S, Volpo S20, Crodacol CS70, Polawax GP 200, Antaron WP-660, DEET and MGK-326. The mixture is stirred and heated. When all ingredients have melted, oxybenzone is added. Heating is continued to 75-80° C. until the oxybenzone is melted then octyl methoxycinnamate is added. Stirring is maintained until the mixture is homogenous and clear.

When both the water and the oil phases are at a temperature of 75-80° C., the oil phase is introduced into the water phase with stirring. When all the oil phase is added, stirring is stopped and the mixture is homogenised for five minutes. Stirring is recommenced and the chelating agent and half of the neutraliser are added followed by the addition of the titanium dioxide. The remaining neutraliser is then added. Stirring is stopped and the mixture is homogenised for five minutes. Stirring is recommenced with addition of moisturiser, emollient and preservative.

If a zinc oxide inorganic sunscreen agent is used, a different thickening system would be appropriate. A cellulose-based thickener such as methyl cellulose, guar gum, sodium alginate and sodium carboxymethyl cellulose could be used, in which case a neutraliser would not be required.

Testing

Formulae 1 and 3 were tested in two ways:

A. Determination of sun protection factor (SPF) and

B. Broad spectrum test.

A. Determination of Sun Protection Factor (SPF)

Principle: The individual sun protection factor, SPF, of a sunscreen product is determined from the minimum erthemal dose (MED) of the skin that has been protected with the sunscreen product and from the MED of an adjacent area of unprotected skin, under specific conditions by means of the following relationship, where the UV source has constant intensity:

$$Sun Protection Factor = \frac{MED for protected skin}{MED for unprotected skin}$$

The sun protection factor of a product is calculated as the arithmetical mean of the individual sun protection factors. MED is defined as the amount of energy from any source required to produce a minimally perceptible redness reaction of the skin.

Test Procedure:

The MED of the (untreated) subject at the test site is first determined using a solar simulator. An experienced tester can often predict a MED for a particularlamp intensity and subject but, where necessary, one or more sets of exposures must be read 16 h to 24 h later to determine the approximate MED without exposing the subject to excessive radiation. Exposures are made on one or more small subsite areas at measured exposure times.

On the basis of this predicted or approximate value, the MED is determined more precisely by a set of exposures which span a dose range of approximately 0.6 to 1.5 of the MED. Usually, these doses are administered the day before the product is tested but they may be administered at the same time. When the doses are administered the day before, the result when read, not only provides the denominator for calculating the protection factor but, when multiplied by the expected or likely value of the product's protection factor, provides an estimate for the longer exposure needed to assess the product.

The product is assessed by exposing a set of small subsite areas adjacent to the untreated areas, after application of the product. Times of exposure are selected to bracket the above estimate. when read 16 h to 24 h later, the MED for the treated skin is divided by the MED for untreated skin to give the protection factor.

The results of the tests on formulae 1 and 3 are shown below in table 2.

TABLE 2

| | | | | MED. | Protected | |
|---|---|---|---|---|---|---|
| | Subject | Sex | Skin Type | (sec) | MED (sec) | SPF |
| Formula 1 | A | F | III | 16 | 496 | 31 |
| | B | M | II | 10 | >341 | 34.1 |
| | C | M | II | 12 | 372 | 31.0 |
| Formula 3 | A | F | III | 16 | >496 | >31.0 |
| | B | M | II | 10 | 341 | 34.1 |
| | C | M | II | 12 | >450 | >37.5 |

Skin Type = I-sensitive, always burns
II-moderate, burns sometimes
III-normal, burns and tans

MED=Minimal Erythermal Dose SPF over 30 was demonstrated in each case.

B. Broad Spectrum test.

There are three alternative test methods of sample preparation and transmittance measurement in the region 320-360 nm of broad spectrum sunscreen products well known to those skilled in the art. The method used by the inventors is the thin film method.

Materials and Equipment:

The following materials and equipment are required: A spectrophotometer capable of determining percentage transmission from 320-360 nm radiation. A quartz cell, with suitable lid, constructed to provide an 8 μm layer of sunscreen product for testing.

Procedure:

Fill the cell with the sunscreen product and determine the transmission of the product from 320-360 nm inclusive. Record the percentage transmission of the product under test from 320-360 nm inclusive.

Results:

The compositions of formulae 1 and 3 did not transmit more than 10% of UV radiation at any wavelength between 320 nm and 360 nm inclusive.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

The claims defining the invention are as follows:

1. A sunscreen composition, including one or more insect repellents, one or more organic UV sunscreening agents and one or more inorganic sunscreening agents, characterised in that the composition includes 3-9% by weight in total of at least two emulsifiers, based on the total weight of the composition.

2. A sunscreen composition as in claim 1 including two or more emulsifiers selected from the group consisting of fatty acid ethoxylates, fatty alcohol ethoxylates, fatty alcohols and blends of fatty alcohol ethoxylates with alkyl phenol ethoxylates.

3. A sunscreen composition as in claim 1, including two or more emulsifiers selected from the group consisting of glycerol monostearate, ethoxy stearyl alcohol, C₁₆₋₁₈ is fatty alcohols and blends of cetostearyl alcohol and PEG stearate.

4. A sunscreen composition as in any one of claims 1 to 3 wherein the inorganic compound is zinc oxide or titanium dioxide, preferably micronised zinc oxide or micronised titanium dioxide, most preferably micronised titanium dioxide.

5. A sunscreen composition as in any one of claims 1 to 3 including N,N-diethyl-m-toluamide and/or dipropyl pyridine-2,5-dicarboxylate as the insect repellent.

6. A sunscreen composition as in claim 1 including by weight, based on the total weight of the composition,
(a) 1-5%, preferably 2-4%, more preferably 3% inorganic compound as a sunscreening agent,
(b) 4-20%, preferably 4-15%, more preferably 5-10% insect repellent and
(c) 3-10% each of one or more organic UV sunscreening agents.

7. A sunscreen composition as in claim 6 wherein the inorganic compound is zinc oxide or titanium dioxide, preferably micronised zinc oxide or micronised titanium dioxide, most preferably mnicronised titanium dioxide.

8. A sunscreen composition as in claim 6 wherein the insect repellent is N,N-diethyl-m-toluamide and/or dipropyl pyridine-2,5-dicarboxylate.

9. A sunscreen composition as in any one of claims 1 to 8 further including
(d) 7% in total of emulsifiers
(e) up to 5%, preferably 1-5%, more preferably 3% film former
(f) up to 0.25%, preferably 0.05-0.25%, more preferably 0.15% thickener
(g) up to 0.3%, preferably 0.1-0.3%, more preferably 0.15% neutraliser
(h) up to 0.3%, preferably 0.1-0.3%, more preferably 0.2% chelating agent
(i) up to 2.5% of at least one of preservative, perfume and moisturiser.

10. The use of 3-9% by weight in total of at least two emulsifiers in a sunscreen composition, based on the total weight of the composition, which includes one or more organic UV sunscreening agents, one or more inorganic sunscreening agents and one or more insect repellents.

11. A combined insect repellent and sunscreen composition including one or more insect repellents, one or more organic UV sunscreening agents and one or more inorganic sunscreening agents, characterised in that the composition includes 3-9% by weight in total of at least two emulsifiers, based on the total weight of the composition.

12. A method of manufacturing a sunscreen composition including one or more insect repellents, one or more organic UV sunscreening agents and one or more inorganic sunscreening agents, the composition being in the form of an emulsion having an oil phase and a water phase characterised in that the water phase and oil phase are prepared and combined to form an emulsion prior to addition of at least one inorganic compound which is used as a sunscreening agent.

13. A method of manufacturing a sunscreen composition including the steps of:
(a) preparing a water phase including water and thickener;
(b) preparing an oil phase including at least two emulsifiers, at least one insect repellent and at least one organic UV sunscreening agent;
(c) combining said water phase and oil phase to form an emulsion; and
(d) adding at least one inorganic compound which is used as a sunscreening agent.

14. A method of manufacturing a sunscreen composition in the form of an oil-in-water emulsion including the steps of:
(a) preparing a water phase by combining water and thickener while stirring and heating,
(b) preparing an oil phase by combining at least two emulsifiers, at least one insect repellent, optionally a film former and at least one organic UV sunscreening agent while stirring and heating,
(c) adding the oil phase to the water phase while stirring,
(d) optionally adding a chelating agent and a neutraliser to the combined water and oil phases; and
(e) adding at least one inorganic compound which is used as a sunscreening agent to the combined water and oil phases while stirring.

15. The method of claim 14 wherein the water phase of step (a) and the oil phase of step (b) are heated to a temperature in the range of 75-80° C. respectively before combining in step (c).

16. The method of any one of claims 13 to 15 wherein at least two emulsifiers are selected from the group consisting of fatty acid ethoxylates, fatty alcohol ethoxylates, fatty alcohols and blends of fatty alcohol ethoxylates with alkyl phenol ethoxylates.

17. The method of any one of claims 13 to 15 wherein at least two emulsifiers are selected from the group consisting of glycerol monostearate, ethoxy stearyl alcohol, C₁₆₋₁₈ fatty alcohols and blends of cetostearyl alcohol and PEG stearate.

18. The method of any one of claims 12 to 15 wherein the inorganic compound is zinc oxide or titanium dioxide, preferably micronised zinc oxide or micronised titanium dioxide, most preferably micronised titanium dioxide.

19. The method of any one of claims 12 to 15 wherein the insect repellent is N,N-diethyl-m-toluamide, dipropylpyridine-2,5dicarboxylate or a mixture thereof.

20. The method of any one of claims 12 to 15 wherein the organic UV sunscreening agent is oxybenzone, octylmethoxycinnamate or a mixture thereof.

21. A sunscreen composition manufactured according to the method of any one of claims 12 to 20.

22. A sunscreen composition, including at least two emulsifiers as hereinbefore described with reference to the examples.

23. A method of manufacturing a sunscreen composition as hereinbefore described with reference to the examples.
